(19) United States
(12) Patent Application Publication
(10) Pub. No.: US 2002/0095075 A1
(43) Pub. Date: Jul. 18, 2002

(54) Photonic molecular probe applications

(76) Inventors: Frank Madarasz, Madison, AL (US); David P. Krivoshik, East Amwell Township, NJ (US)

Correspondence Address: MATHEWS, COLLINS, SHEPHERD & GOULD, P.A., 100 THANET CIRCLE, SUITE 306, PRINCETON, NJ, 08540-3674, US

(21) Appl. No.: 10/081,363

(22) Filed: Feb. 21, 2002

Related U.S. Application Data
(63) Continuation-in-part of application No. 09/860,362, filed on May 18, 2001.
Application No. 09/860,362 is a continuation of application No. 09/249,677, filed on Feb. 12, 1999, now Pat. No. 6,236,870.
Application No. 09/249,677 is a continuation-in-part of application No. 08/858,260, filed on May 19, 1997, now Pat. No. 5,871,442.
(63) Continuation-in-part of application No. 09/502,248, filed on Feb. 17, 2000.
(60) Provisional application No. 60/024,727, filed on Sep. 10, 1996.
(60) Provisional application No. 60/120,309, filed on Feb. 16, 1999.

(51) Int. Cl. A61B 5/00

(52) U.S. Cl. 600/310; 600/316; 356/364

(57) ABSTRACT

An opto-electronic device which utilizes a band of polychromatic light for quantitative analysis of a target molecule within a mixed specimen, where a selected optical path is used to measure the reference molecule having a predefined, known or determinable value, and a second measurement is made through the selected optical path, so that the optical distance of the selected path is corrected for by use of the first reference molecule measurement.

### CROSS REFERENCES TO RELATED APPLICATIONS

This application claims the benefit of U.S. Provisional Application Serial No. 60/270,332, filed on Feb. 21, 2001 and is a continuation-in-part of co-pending U.S. patent application Ser. No. 09/860,362 filed on May 18, 2001, which is a continuation of now issued U.S. Pat. No. 6,236,870, which is a continuation of now issued U.S. Pat. No. 5,871,442, which claims the benefit of U.S. Provisional Patent Application No. 60/024,727 filed on Sep. 10, 1996. This application is also a continuation-in-part of co-pending U.S. patent application Ser. No. 09/502,248 filed on Feb. 17, 2000 which claims the benefit of U.S. Provisional Application Serial No. 60/120,309 filed on Feb. 16, 1999.

### FIELD OF THE INVENTION

This invention relates generally to the quantitative determination of optically active substances, and more particularly to using polychromatic light for quantitative determination of optically active substances.

### BACKGROUND OF THE INVENTION

Monitoring the levels of various chemical agents in human serum and in the environment is important in the treatment and control of diseases as well as in law enforcement.

Diabetes mellitus is a chronic disease which requires monitoring of blood glucose for proper control. Repetitive determination monitoring of blood glucose is necessary to adequately provide controlled insulin dosing. Currently accurate monitoring is available only by taking and analyzing a blood sample. This invasive procedure is time consuming and not practical for continuous monitoring.

Measurement procedures in law enforcement, including those for intoxication with alcohol, currently utilize indirect tests such as a breath analyzer, motor coordination tests, or require a blood sample. The drawing of a blood sample is an invasive technique which generally necessitates that the blood sample be sent to a laboratory for analysis. Delays in drawing the sample reduce the utility of the test results.

Miniaturized technologies capable of continuous, real-time sensing of chemical/biological warfare agents such as nerve gases, smallpox and anthrax are critical in combating terrorism.

Emergency medical personnel need to be able to immediately, accurately and reliably assess patients' blood levels of both illicit and licit drugs and make confident, correct clinical treatment decisions.

Compliance of the patients with treatment regimes can dramatically improve, and relevant serum level diaries can become easy to maintain by patient or physician where appropriate (e.g., lithium carbonate, tegretol, sodium divalproex, glucose, various hormones, etc.) with an accurate non-invasive quantitative analysis device.

The dangers of contacting blood from an individual who is HIV positive or who has Hepatitis are well known. Extreme caution must be taken in drawing and processing the blood samples. Permission of the individual or a court order may be required to obtain the blood sample. Typically, the sample must be drawn by a medically qualified individual. Also, the venipuncture of an immune-compromised individual is, in itself, a risk to that person.

### SUMMARY OF THE INVENTION

In accordance with the present invention, there is provided an opto-electronic device which utilizes a band of polychromatic light for quantitative analysis of a target molecule within a mixed specimen.

### BRIEF DESCRIPTION OF THE DRAWINGS

A more complete understanding of the present invention may be obtained from consideration of the following description in conjunction with the drawings in which:

FIG. 1 is a block diagram of a first illustrative embodiment of the Photonic Molecular Probe;

FIG. 2 is a block diagram of a second illustrative embodiment of the Photonic Molecular Probe; and,

FIG. 3 is a flow chart of data acquisition and analysis.

### DETAILED DESCRIPTION OF VARIOUS ILLUSTRATIVE EMBODIMENTS

The present invention Photonic Molecular Probe (PMP) is a non-destructive/non-invasive monitoring device, capable of probing and unambiguously identifying quantitatively a target molecule within a mixed specimen. Because the operational capabilities incorporate several physically distinct modes of operation the Photonic Molecular Probe has a myriad of potential applications.

Although the Photonic Molecular Probe is particularly well suited with monitoring blood constituents such as alcohol, glucose, and triglycerides, and shall be described in this application, the present invention is equally well suited for use in food inspection, plastic waste disposal and continuous alcohol monitoring in brewing vats. The present invention is equally well suited for other production and process operations in which continuous quantitative analysis is advantageous.

While one embodiment of the Photonic Molecular Probe is non-invasive and employs NIR (near Infared) light, it does NOT rely on linear absorption. The Photonic Molecular Probe uniquely exploits the chiral asymmetry, geometrical, properties of the target molecule, such as glucose, which influences the light in a process called Circular Dirchroism (CD), chiral absorption. The result is a much richer source of information leading to the unambiguous structural identification of the target moleculr. Concurrent with CD is another chiral process called Optical Rotary Power (OR). It should be noted, that any molecule or substance, which exhibits CD and OR, is described as Optically Active.

Organic molecules, such as glucose, are structured in a spiral form and have a definite helicity or handedness. It is this helicity which gives a molecule its ability to rotate the polarization of the incident light.

The magnitude of the angle through which the polarization direct rotates is proportional to the square of the frequency of the incident light. It also is a strong function of the type of material or molecular structure being irradiated. This functional dependence on physical properties of the medium manifests itself in the difference of the indices of refraction for right-handed and left-handed polarized light, which make up linear and elliptical polarization states, producing a relative phase shift between the two. Linear and elliptically polarized light are a result of the superposition of two circularly polarized beams of light—one rotating clockwise and the other counter-clockwise.

If, on the other hand, there is a difference in the absorption between the right-handed and the left-handed circularly polarized light making up the polarization state, the phenomenon of Circular Dichronism exists. For example if the polarization of the light irradiating the sample were purely elliptical, not only would the ellipse rotate, due to the OR, about an axis parallel to the direction of propagation of the light but also the ellipse would distort, that is its eccentricity would change.

A quantitative basis for an understanding of how polarized light interacts with molecular species having a definite helicity or handedness may be obtained from consideration of Appendix A—Summary of Polarized Light, and of Appendix B—Opitcal Rotation and Circular Dichroism of U.S. Pat. No. 5,871,442. Optical rotation and circular dichroism are two opto-electronic processes essential in numerous applications of the device. A review of these appendices should make the following discussion of opto-electronic processes and their relationship to the device more transparent.

The Photonic Molecular Probe is designed to optimize data collection and device miniaturization. It uses state-of-the-art optical and electronic component technology as well as sophisticated data reduction techniques.

The Photonic Molecular Probe operates in a wide spectral region, including, but not limited to, Long Wavelength Infrared to Short Wavelength Infrared and Ultra-Violet, using an elliptical/partially polarized polychromatic (sometimes referred to as chromatically polarized) radiation source. A variety of opto-electronic processes, fundamentally corresponding to basic scattering, processes are utilized to identify the signature and concentration of various target molecules within a mixed specimen with a minimum of data reduction, yielding a highly accurate and cost effective analysis.

The device incorporates advanced designs for the optimization of data collection and device miniaturization, state-of-the-art optical and electronic component technology as well as sophisticated data reduction techniques. Target molecules can be any of a variety of illicit or abused drugs, licit drugs, blood chemistry profile components, hormones, white blood cell counts, red blood cell counts and morphology, as well as numerous other substances. Illicit or abused drug target molecules can include amphetamines, barbiturates, benzodiazepines, cocaine, methadone, opiates, phencyclidine, propoxyphene, secobarbital, tetrahydrocannabinol as well as numerous other illicit drugs. Target molecules for classes of toxic assays of licit drugs can include non-steroidal anti-inflammatory agents (NSAIDs), tricyclic antidepressants (TCAs), anti-psychotics, analgesics, antihistamines, anti-seizure drugs, anti-manic drugs, methanol, mathaqualone, anti-coagulants, anti-hypertensives, salicylate, strychnine, anti-asthma medications, cardiac medications, antibiotics, anti-virals as well as numerous other licit drugs. Blood chemistry profiles present many potential target molecules including albumin, alkaline phosphatase, total bilirubin, calcium, chlorine, cholesterol, creatinine, glucose, lactic acid dehydrogenase, potassium, total protein, glutamic oxaloacetic transaminase, SOOT, SGPT, sodium, triglycerides, urea nitrogen, uric acid, carbon dioxide and numerous others. Hormone tests for steroids such as at athletic competitions, human chorionic gonadotropin (HCG) for pregnancy and precancer tests, T-3, T-4, thyroid stimulating hormone (TSH) as well as other hormones can be target molecules.

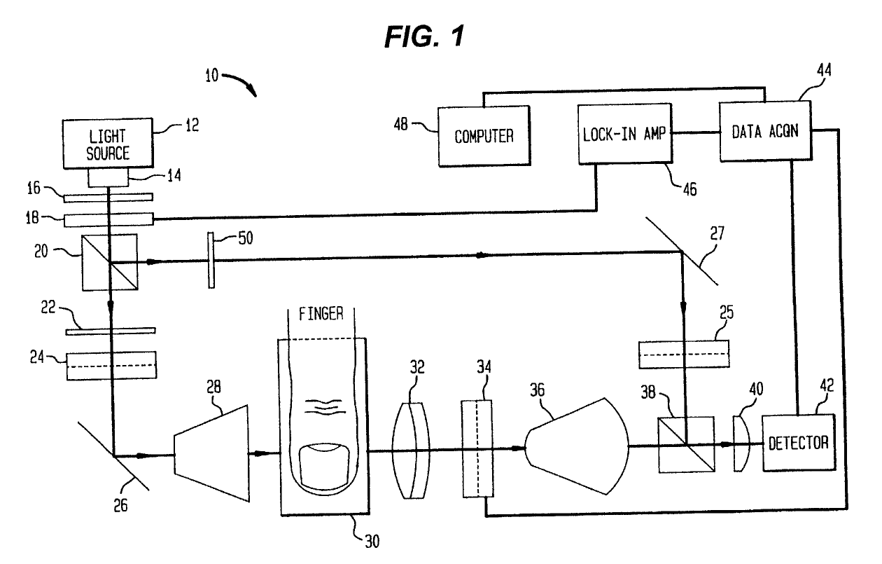

Referring to FIG. 1 there is shown a block diagram of a first illustrative embodiment of the present invention Photonic Molecular Probe 10.

A light source 12, such as a tungsten filament lamp, is used within an envelope with an internal reflector behind the envelope, similar to a sealed beam head lamp. The envelope contains a halogen gas, usually a mixture of an inert gas such as xenon, and a fluoride or chloride bearing gas. The filament of the light source 12 is typically heated to approximately 2900K and emits, by blackbody emission, in the range 320-2500 nm. For wavelengths longer than about 2500 nm a silicon carbide globar lamp at 1500K is used.

The halogen gas mixture is ionized at the high temperature by the filament, and emits short wavelength spectral lines characteristic of the ionized elements similar to a conventional mercury-inert gas fluorescent lamp. The gas also helps stabilize the temperature effects. With a specialty lamp consisting of both tungsten-halogen and florescent characteristics an advantage in calibration could be realized.

A collimating lens 14, may be a part of the lamp, which contains the light source 12, or may be optically coupled to the lamp. Lamps are available with either focusing or collimating lenses, and internal reflectors which provide a degree of elliptical as well as linear polarization (partial polarization).

A color filter wheel 16 can be used to obtain shorter band-pass frequencies for analyses simplification. The color filter wheel 16 is optically coupled to the collimating lens 14.

Choppers are used in spectrometers principally for one of two reasons: stray light control and double beam operation. In the Photonic Molecular Probe 10 a chopper 18 is optically coupled to the color filter wheel 16. The chopper 18 is electronically coupled to a Lock-in Amplifier 46.

For stray light control the location of the chopper 18 allows timing of a lock-in amplifier 46 to select data which is highly immune to extraneous light. Frequency shifts due to the position of the polarizing elements, and the polarization shift of the selected wavelength, provide quantitative data as the target molecule interacts with the light from the polychromatic source 12. This data is a complex, superimposed non-imaging spectrum, which contains information about retardation, wavelength and amplitude. The use of the chopper 18 can impede the Photonic Molecular Probe's operation by limiting the speed of data collection. However, proper enclosure designs reduce stray light, reflections, and external illumination.

A double beam operation is obtained by inserting a second sample holder between mirror 27 and polarizing element 25, and also replacing polarizing beam-splitter 20 with a reflective version of chopper 18 mounted at 45 degrees to the path between the collimating lens 14 and a half wave plate 22. This unit would be similar to a conventional chopped double-beam instrument. In addition, if desired, the double beam instrument allows for a self calibrating feature by a real-time comparison of known to unknown concentrations of the target specimen: i.e. self-calibration. While double beam operation is useful for an absorbance or transmittance device, timing options of other movable elements offer better choices for a polarization device.

A polarizing beam splitter 20 is optically coupled to the chopper 18. The operation of the Photonic Molecular Probe requires partially polarized polychromatic light to permit markers for the transform analysis. By multiple reflections of a polychromatic beam each frequency becomes partially polarized with a slightly different angular dependence resulting in a series of markers distributed on an essentially elliptical envelope. A higher degree of polarization can now be obtained by the use of polarizing elements which are minimally affected by lamp aging. By using a broadband beam splitting polarizer a relatively unpolarized light source is split into the S and P polarization states at a 90 degrees exit angle. Additional options include the use of separate polarization devices, such as dichroic polarizers, which are selective at certain wavelengths due to the anisotropic material used.

A half wave plate 22 can be used to control the intensity of the beam to avoid saturation of the detector during calibration or measurements of more nearly transparent materials. The half wave plate 22 is optically coupled to the polarizing beam splitter 20.

A quarter wave plate 50 is used in the reference beam path to control the ellipticity of the beam and also may be used for interference alignment if coherent light is used. The quarter wave plate 50 is optically coupled to the polarizing beam splitter 20.

A polarizer element 24 is optically coupled to the half wave filter 50. The polarizer element 24 is movable and may be dichroic to permit frequency (i.e. wavelength) dependence. The polarizing element 24 is stepped once as an analyzer 34 is incrementally rotated to at least some multiple of 90 degrees. This allows the wavelength dependence of the eccentricity of the polarization ellipse of the partially polarized light through the polarizing beam splitter 20 to be utilized without bandpass filters. An alternative is to use a set of filters at the color filter wheel 16.

If a greater portion of the S polarization state is selected at the polarizing element 24, then the analyzer 34 can be set to transmit the P polarization state to achieve interference at a beam combiner 38.

A first flat mirror 26 is used to direct the partially polarized light to a finger cell 30 through a collimator 28 without chromatic aberration. The reflection at the first flat mirror 26 rotates the polarization 45 degrees counter clockwise while retaining the polarization markers.

A second flat mirror 27 is used to direct the optical output of the quarter wave plate 50 to a second polarizing element 25. The second polarizing element 25 is optically coupled to the beam combiner 38.

A collimator 28 minimizes extraneous scattered light and expands the beam to one of a larger cross-section and lower amplitude, which reduces heating and increases optical interaction with the finger fluids. The collimator 28 can be a reflective device to minimize chromatic effects. The chromatic shift is fixed and can be dealt with in calibration if a lens arrangement is used.

A finger cell 30 can be constructed with cylindrical lenses to optimize the beam path through the finger. While this will affect the polarization it can be used advantageously if a conjugate cylindrical lens is used on the exit side. The light into the finger is partially scattered upon exit. This in turn gives a logarithmic amplitude dependence of light transmitted with respect to cell path length through the sample. Hence by linear collimation and recollection of the scattered light, these lenses minimize problems with varying finger sizes. The intensity of light through the finger is a function of the path length and the concentration of any analyte should be consistent with the normalized intensity. The distance between the cylindrical lenses can be measured as part of calibration to a particular patient. Normalization to other standards, such as water, can also provide accurate calibration. In this case, the amount of analyte divided by the water signal will provide the normalization.

For other embodiments of the present invention the finger cell 30 is generally a specimen cell which is adapted for receiving a particular mixed specimen. While the present invention is particularly well suited for use in non-invasive analysis it is equally well suited for use in the analysis of mixed specimens which may have been collected by an invasive technique as well as specimens which originated from other sources such as a laboratory, production environment or a process operation. Mixed specimens collected by traditional invasive techniques into a vial may be placed into a suitably adapted specimen cell for analysis. The specimen cell is adapted for placement within an operation, such as in a process operation, where continuous quantitative analysis is advantageous and may permit the mixed specimen to flow through the specimen cell. This is ideally suited for use as an implanted sensor as well as for suspension of the sensor is a process vat or conduit.

A condensing lens 32 is optically coupled to the finger cell 30 and collects the dispersed light without adding additional depolarization.

The analyzer 34, which is optically coupled to the condensing lens 32, is a movable non-dichroic polarizing element. It is used to track signal variations derived by light intensity versus angular position. The light in this case is partially polarized in a wavelength dependent manner by both the light source 12 and the polarizer element 24. A complete signal is obtained by incrementally moving the analyzer 34 (or electronically scanning a spatial light modulator) through at least 180 degrees. Next, the polarizing element 24 is incremented by a predetermined value, preferably small, to provide a shift in the dichroism and polarization. The analyzer 34 is again scanned at least 90 degrees, or preferably 180 degrees, and the data compared to the previous data.

The difference of data collected when the analyzer 34 is rotated the same 90 degrees (starting position the same), and the polarizing element 24 is stepped, yields phase sensitive differences due to polarization and is influenced by wavelength sensitive dichroism. Another mode of operation is to spin the analyzer element 34 and synchronize an oscilloscope to sweep each half rotation. This will provide a stationary signal. Now the polarizing element is incremented to observe the difference. The data is stored digitally, in columns and rows, for further analysis employing various digital filters. In addition, interferrometric data will be produced which can be analyzed with frequency transform methods.

This process can be repeated for positions of the polarizing element 24 to 90 degrees to complete the spectrum. If an opaque beam block is placed between the second mirror 27 and the second polarizing element 25, and no finger is present, then a characteristic signal is achieved for the analyte path. Alternatively, if an opaque block is placed in the cell, then a source calibration is achieved.

A beam reducer 36 can use reflective Cassegrain optics to reduce chromatic shift. Polarization, albeit fixed, is dominant over a lens. The beam reducer is optically coupled between the analyzer 34 and the beam combiner 38.

The optical path between the collimating lens 14 and the analyzer 34 is now crossed with the path between the collimating lens 14 and the second polarizing element 25, producing an initially dark signal at the beam combiner 38, without a chiral sample with no chirality in the finger cell 30. This is because it should be easier to detect small increases in the intensity of light in a dark environment than small decreases in the intensity of light in a bright environment. Note the 45 degree rotation. Furthermore, although the source may not be completely coherent, a coherence path length does exist.

Regardless of whether phase information is collected at separated frequencies by the polarizer element 24, or by any other means, whole blood will yield a very complicated spectrum comprised of a system of superimposed spectra. The polarizer detects primarily phase information.

By using a dual path approach, instead of the partially polarized single path device, two very important goals are achieved.

First, a reference signal is always available for calibration, thereby allowing time-independent calibrations. Furthermore, independence from calibration specific to a given patient becomes a reality, especially with a finger cell 30 that is adjustable.

Second, overlapping Lorentzian absorption bands generate a complex spectrum which can be expressed as an infinite sum of sine and cosine terms. Additionally, information from intensity versus polarizing analyzer position (with sample present) is continuous, and will therefore also have phase sensitive information. One way to handle this massive amount information is to subtract successive intensity data collected throughout the rotation of the analyzer 34 at discrete steps of the polarizer element. This data is combined at the beam combiner 38 with the conjugate polarized original light (S vs. P), yielding interferometric or retardation data. This provides for a real Fourier Transform (or other frequency dependence) instrument. The analysis can be handled similarly to known FTIR spectrometer methods wherein the spectrum is calculated from the interferogram comprised of intensity information from the spectrum of the source minus that of the sample. Since the light is not monochromatic the interferogram is now no longer a simple cosine function.

A forward difference approach may be used to remove large spectral changes and allow observation of the smaller spikes. The application of wavelet theory can be very useful, however the complexity of this task can be reduced by subtraction of intensity data collected at different increments of the polarizer element 24 while rotating the analyzer 34. This then permits true wavelength dependence to be observed and retarded for frequency analysis.

If the polarizing analyzer is rapidly rotated in a continuous fashion, the retardation intensity at the output of a detector 42 versus the position of the polarizing element 24 is a shifting spectrum. The sample information is then derived from successive subtractions of intensity throughout the rotation of the analyzer 34, in any multiple of 180 degrees, plotted against the angular position of the polarizing element 24. As mentioned previously, direct viewing with an oscilloscope is possible by synchronizing in rotation multiples of 180 degrees for each sweep of the oscilloscope.

If the pixel size of the detector 42 is less than the beam diameter from the beam combiner 38 then a condensing lens 40, or focusing lens, can be used by optically coupling the condensing lens 40 between the detector 42 and the beam combiner 38.

The detector 42 can be a single element high speed photo detector diode or photocell having an array of detectors with separate wave length response. A data acquisition system 44 is electrically coupled to the detector 42 and the analyzer 34. An analysis computer 48 is electrically coupled to the data acquisition system 44. For high resolution accuracy a lock-in amplifier 46 is electrically coupled to the chopper 18 and the data acquisition system 44.

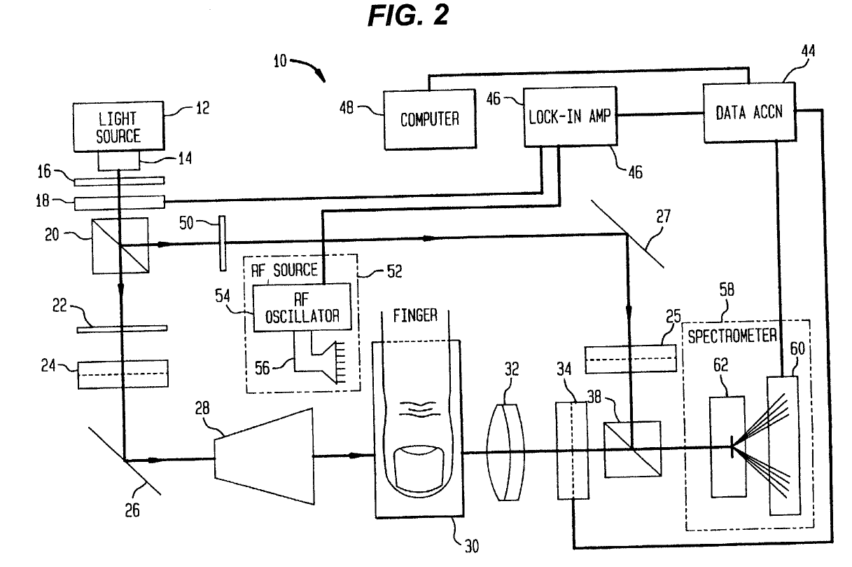

A more versatile instrument is possible by using a small monolithic spectrometer and an array diode to obtain color (or spectral) information similar to FTIR instruments. Referring to FIG. 2, a second illustrative embodiment of the present invention Photonic Molecular Probe which is specifically designed to make possible the assay of substances with no naturally occurring inherent chirality is shown. In this way a snapshot of the spectrum is obtained each time an event signal is triggered. Elements that have similar functions as those is FIG. 4 have been assigned the same reference number and are not again described in detail. For example, each time the polarizing element 24 is incremented, such as when the analyzer is at zero and 180 degrees, a spectrum can be saved. These spectra can then be added for small ranges of the polarizing element 34 known to be specifically active for a particular analyte. Since the spectrum is direct and obtained by a detector array 60 it is not necessary to decompose the retardance data to obtain sample information. An RF source 52 causes directional alignment of certain achiral molecules to assist in detection and identification.

The RF source 52 is electrically coupled to the lock-in amplifier 46 and coupled to the specimen in the finger cell 30. RF source 52 is comprised of an RF oscillator 54 and a resonant coupler 56, such as a coil or dichroic antenna, to excite the molecules during measurement. The output of the beam combiner 38 is optically coupled to a spectrometer 58. The spectrometer 58 is comprised of the detector array 60 and a beam separation element 62, such as a monochrometer. The spectrometer 58 is electrically coupled to the data acquisition system 44.

Additionally, since noise is random, the noise is reduced by the square root of n samples taken and added over the limits of collection and computation. If for example, in the first embodiment of the Photonic Molecular Probe, 3600 data points are collected in the 360 degrees rotation of the analyzer 34, and polarizer element 24 is incremented one degree for each revolution of the analyzer, for 360 degrees, then about 1.3 million data points are rapidly achieved at nominal collection rates. This data can be plotted as the transform of the difference in any two rows of intensity versus position of the analyzer for two corresponding positions of the polarizer element 24. This can alternatively be performed with the second mirror 27 turned or blocked so as to produce a single pass instrument.

With this approach much more versatility is possible from the double pass than from a single pass device. Consider observing retardation data with the second mirror 27 in place. Assume 3600 data points are observed with a Fast Fourier Transform (FFT) taken of the difference of successive rows as the polarizer element 24 increments. For 360 values of the polarizer element 24 then 1.3 million values of retardation are decomposed to a transform spectrum, noting that this spectrum is not a discrete wavelength spectrum but an interferogram source spectrum none the less. A presentation of intensity transform vs. position of the analyzer 34 and the polarizer element 24 is a 3D graphical representation of the chiral (and other absorption) interference due to the molecular activity in the finger cell 30. In order to excite polar molecules and molecules of low chiral activity (e.g. some substances other than glucose), it is possible to add the RF source 52. For analysis of very light elements direct current electrophoresis and/or a large magnetic field may also be required, utilizing NMR procedures.

Most FFT procedures use 2ⁿ data sets where the abscissa (or rotational position in this case) may be generated once rather than measured. In other words, the angular position is repeated and so it becomes necessary only to count the output control steps rather than measure the actual position of the rotating device. Therefore an analog signal marked by steps of the polarizing element 24 can be stored digitally and, if the rotation is fairly smooth, very good values for angular position could be substituted. With this process values of rotational position not related to direct measurements can be generated: i.e. 4096 data points per revolution (2¹²) for instantaneous response could be synthesized in real time. If it is not possible to achieve the needed rotational correlation exactly enough each time then a high resolution index device is used determine the position of the analyzer 34 and the data acquisition system 44, and the analysis computer 48 becomes the limiting factor of the speed of operation. The lock-in amplifier 46 may be omitted in a portable or lower cost version, however this will be at the cost of some reduction in accuracy. Since the data collection at the analyzer 34 is very position (and environment) dependent, the lock-in amplifier 46 needs to synchronize the light blocking function of the chopper 18 and the data acquisition system 44 at rather high speed, especially in a research or laboratory version of the present invention Photonic Molecular Probe where maximum performance is required. Spatial light modulator active optical devices exist which may be substituted for the chopper 18 or the analyzer 34 but presently remain expensive although experimental versions are available.

Other data reduction procedures can be utilized, such as wavelet theory or boxcar methods, which will correlate the small difference signals ideally. Other methods of analyzing blocks of data for optical spectrometers and other instruments are well known to those skilled in the art.

### Data Processing Requirements

The specific data processing requirements for the Photonic Molecular Probe will depend upon the sophistication of the device model which can be highly tailored to the specific needs at hand. A significantly degraded (i.e. simplified) version of what is to follow may be sufficient for most applications.

The data collection system in its most general form may be envisioned as consisting of a two dimensional sensor array coupled to the sensors for the angular positions for one or two optical elements in the instrument, all synchronized to the data processing clock either directly or indirectly through another intermediate clock. The sensor array may be frequency (i.e. color) sensitive, and this may prove to provide additional substance characterization information, and possibly further reduce the sophistication needed in the data acquisition of the other parameters mentioned above. Simplifications in this most general detection system may include collapsing the array to a line or a point, needing only one instead of two positionable optical elements, in addition to others.

While the data processing is done completely on board the Photonic Molecular Probe, the capability of interfacing with external computers exists. The sensed signal is represented by a two dimensional intensity array which, when combined with the information from the polarizing analyzer, contains the crucial angular information previously discussed at length. This signal is highly noise immune. One important observation about this configuration is that the techniques of wavelet transforms and Hadamard transforms may be of great use. This results in a significant reduction in the required computational power, and therefore enhanced possibilities for miniaturization, and portability, and substantially reduced costs. One example of this is in dealing with the two dimensional optical display, in which case the anti resolution, orthogonal, and biorthogonal wavelet transforms can prove quite useful.

Essentially wavelet expansions are just another form of convenient expansions, not unlike Fourier or Hartley transforms, of members of a function space. One unique characteristic is that wavelet transforms map scalar variables into a two dimensional complex domain, and it is this characteristic from which much of their usefulness derives.

The specific wavelet construct to be used in the signal processing will depend on the target molecule and the data acquisition. A general approach to the Photonic Molecular Probe signal processing/discrimination is based on a well known conditional probabilistic reduction method known as Bayes' Rule. The uniqueness of employing the Bayesian method with the Photonic Molecular Probe lies in the fact that very precise physical models can be built directly into the method. For example, atomic form factors, which characterize the electronic structure of various target molecules, can be used for cellular image enhancement. Structure factors (analogues to those used in x-ray diffraction) are another example which characterize the signature of various concentrations of target molecules in solution. The physical models can be all empirical, all theoretical, or a combination of both.

Detailed information on the functioning of the Photonic Molecular Probe can be found in U.S. Pat. Nos. 5,871,442 and 6,236,870 which are incorporated by reference as if set out in full.

Both for the above example using forward differences, and for any other data treatment version, the overall analysis is to solve or invert these equations in order to obtain c. This is most effectively done by the Bayesian method as outlined below. In this formulation the optimum amount of information, along with its associated reliability, may be extracted from each data set.

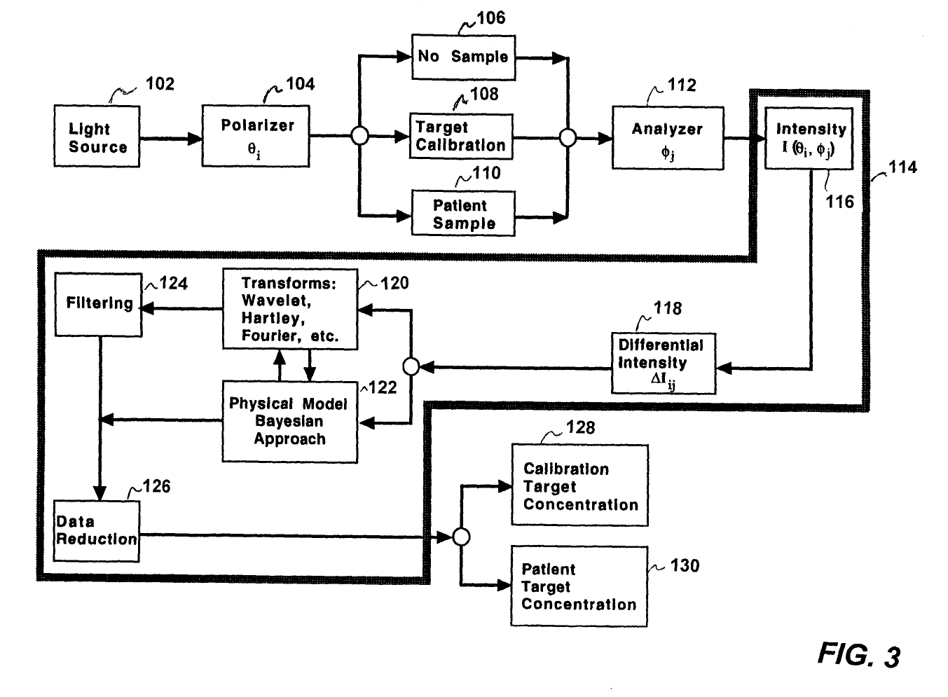

The general approach to the data acquisition and analysis, using the Photonic Molecular Probe device, is shown in flow chart form in FIG. 3. Light source 102 is optically coupled to polarizer 104. Polarizer 104 is optically coupled to an empty cell 106, a calibration target cell 108 and/or a target specimen cell 110, the optical output of which is coupled to an analyzer 112. The analyzer 112 is coupled to a data acquisition and analysis device 114 which is comprised of an intensity determination system 116, coupled to a differential intensity system 118, which is coupled to a transform system 120 and a physical model 122. The Transform system 120 is coupled to a filtering system 124. The physical model 122 and the filtering system are coupled to a data reduction system 126. The Data acquisition and analysis device 114 provides a calibration the target concentration 128 and the specimen target concentration 130.

General Scheme for Frequency Selection-A Phenomenological Approach

Without the first round of data it is of course impossible to set forth a detailed frequency selection scheme: if that were possible we would not need the first round of data. However, certain guiding principles may be enumerated that will guide any specific selection of these frequencies. For illustrative purposes, glucose is cited to be the target molecule in what follows. However, the general procedure of frequency selection for any molecule of interest is the same.

1. Assume that all centered frequencies (i.e., centered within a particular narrow band width to be processed by the Photonic Molecular Probe) within the physiological window, 600 nm to 1200 nm, have equal a priori probability of being relevant/necessary/sufficient for the unique determination of glucose under all possible physiological conditions. Ideally the final signature will be exactly that—necessary and sufficient. Depending upon light sources available, or the ease/economy with which certain frequencies may be generated within this window, three or four frequencies are selected which sample the window in some approximate fashion. In other words, try to avoid all three or four frequencies being, say, below 800 nm, or from any narrow band. With this choice fixed, the instrument is then stabilized and calibrated in the usual fashion: no target, empty cuvette, distilled water, calibrated glucose solutions with and without various contaminants, in vitro blood work, and finally in vivo studies.

2. If at any of these stages of increasing complexity it is found that the introduced confounding elements (e.g. contaminants) make the chosen signature insufficient to uniquely identify glucose, then the character of the indeterminancy (degeneracy) should be examined and the nature of the optical probe is changed. This change can be the addition of more frequencies, the deletion of frequencies originally chosen, frequency shifts, etc.

3. The above process, described in steps 1 and 2, is iterated through increasingly complex environments, including any that are likely to be met in clinical practice. In this way the initial optical signal is built to have the minimum complexity to completely determine glucose in any environment.

A comprehensive discussion of signal processing and the Bayesian method may be found in U.S. patent application Ser. No. 09/502,248 which is incorporated by reference as if set out in full.

The Photonic Molecular Probe may be used to sense a target specimen contained within blood by first obtaining a reference measurement of a body part (finger, ear lobe, etc.) that is in a compressed state, where blood flow is restricted and blood squeezed out, then taking a second measurement after blood has returned with only a nominal pressure on the body part. The difference between the two measurements will correspond to the measurement of the blood, which has returned.

The Photonic Molecular Probe enables the practical implementation of artificial secretory organs, implanted medical delivery systems and external medical delivery systems. External delivery can be accomplished by skin absorption, injection, air injection, oral application, nasal application, rectal application, and other means which are known to those skilled in the arts.

The implantation of a sensor typically was limited by the build up of deposits and body tissues on the sensor. However the application of a ratio sensor utilizing the Photonic Molecular Probe technology results in independence of the actual path length. In one example a first reference target is measured having a predefined, known or determinable value, a second measurement is made through the same path. The actual distance of the path is corrected for by use of the first reference measurement. While the path may consist of different body materials and thus have different references, the squeeze method described above, implantation into the blood stream, or the selection of suitable references, which correspond to the desired target within the different body materials, may be selected.

The use of the Photonic Molecular Probe may be easily extended to other systems, where the build up of deposits on the actual sensors occurs as a result of the material that the sensor is placed in. Again the application of a ratio can be used to either calibrate the actual sensor path or to continuously adjust the sensor path.

When the Photonic Molecular Probe is calibrated, the effects of varying finger thicknesses, color, structure, and contamination must be accounted for. A description of some of the calibration methods follows:

Glucose Calibration:

The secondary path allows for a real-time comparison of known to unknown concentrations of the target specimen. In other words, placing a calibration sample in the path and coding the appropriate software into the Photonic Molecular Probe, allows for the possibility of self-calibration, which is essential for a diagnostic instrument. In addition, the secondary path is a reference path. It is made up of a ¼ wave plate, a reflecting mirror, and an incremental stepwise rotating polarizer. The quarter wave plate is used in the reference beam path to control the ellipticity of the beam and also may be used for interference alignment if coherent light is used.

Finger Thickness/Structure:

From about 760 to 1200 nm the absorbance due to water is about one to two orders of magnitude smaller than that of pure glucose, with some stronger water interactions at about 900 nm in tissue. The “therapeutic window” is centered around 1.0 micron. Absorbance due to water is still only about 5-10% of the total light directed to a surface of tissue, such as the finger, in this NIR range, and, indeed, this effect may become the calibration means. In fact, this is the approach that reference 8 uses in making oximetry measurements through a human finger.

Color:

Should have no effect on calibration. It may cause some unwanted absorption and reflection, but again preliminary testing indicates it should not be a problem.

Contamination:

Certainly whatever part of the body which the measurement is being made on should ideally be as clean as possible and indeed it may be good practice to cleanse that part before the measurement is made. However, under typical operation the polarized IR light is not be disrupted from adequate transmission by small amounts of grease or dirt.

Specific Calibration Per Person:

If the studies with the reference channel show that a properly designed reference sample can compensate for finger or tissue effects we don't presently know about, then no specific calibration may be needed. Second if the analytic glucose in solution studies show that the machine calibration is valid for tissue, we are home free. Worst case would be an infrequent calibration against an invasive method.

Issues in General:

Blood glucose vs. interstitial fluid glucose. Recent research corroborates the fact that the intracellular and interstitial concentrations equilibrate with extreme rapidity, especially for glucose, so the precise ratio of these two compartments traversed by the beam is irrelevant. This is true for the upper part of the body or where blood perfusion is rapid: eyes, ear, tympanic membrane, forearm, fingers and hands. The delay is still on the order of 4 min lag behind on increasing glucose and 4 to 8 min behind when the glucose level is falling. The latter is very useful to give extra time to detect falling glucose levels before they become critical—the under the skin area is emptied first.

Calibration begins with developing a minimal signature in vitro: e.g. glucose in distilled water. Various contaminants may be added to that in vitro experiment to develop the signature further and ensure correlation with concentration of only glucose. After that in vivo runs are done and compared to known accepted assay techniques. Again, the signature is further developed (lengthened) if necessary until satisfactory clinical correlation is achieved.

Small variations in the temperature of the body part in which the measurement is being made should have no effect. A change of temperature from 98 F. to 102 F. is approximately 2×10⁻⁴ eV. This is hardly enough to affect the vibronic states—it certainly will not affect the pure electronic states, which have energy on the order of eV.

The Photonic Molecular Probe can measure and track the changes in glucose levels in a subject undergoing rapid changes up or down in glucose level. In order to understand how the device be configured to accomplish this, the answer requires breaking the question into several parts:

1. When the glucose level in a subject rapidly changes, where do these changes appear in the vascular and tissue structures?

2. What is the relationship between the changes monitored in selected tissues and the vascular glucose level in the body?

3. How can the Photonic Molecular Probe device be configured to monitor those changes?

Glucose levels in the body are traditionally monitored by taking two types of blood samples. One is from a venipuncture in the arm, and the other by taking a shallow lance, fingerstick on the hand. In the first case, the blood comes from a major vein in the arm, which carries a slightly depleted load of glucose since the arterial system has perfused the body with nutrients. The venous system carries out the wastes and the leftover nutrient supply. The fingerstick sample is from the capillary bed, which lies near the base of the epidermis layer of skin. The capillary blood exchanges nutrients with the interstitial fluid, which surrounds the tissue cells. In animals and humans, this capillary blood is the primary source of cellular nutrients.

In the typical human body, the upper parts of the torso are more rapidly perfused with blood than the lower parts. In general, the head, arms, hands, and chest at or above the heart level are well supplied with blood while the lower chest, abdomen, and legs are less rapidly perfused by blood. For clinical purposes, glucose levels in blood sampled from the arm or finger represent the best estimate of the blood glucose for the body.

Much literature has been published in the last 10 years on the use of implanted glucose sensors, microdialysis or ultrafiltration sampling loops, and sampling interstitial fluid from various locations in animals or humans. Briefly, if the sampling point is in the rapidly perfused portion of the body and near a capillary bed of microvessels, the time it takes for capillary blood glucose to diffuse into the interstitial fluid is on the order of 4 to 8 minutes. Interstitial fluid samples from tissues such as the dermis layer (below the epidermis), or from the fatty tissues in the abdominal area have shown that the glucose exchange time lag is even longer.

There are several clinical situations for which blood glucose samples are taken, and the relationship to glucose in the interstitial fluid would be different according to the situation.

First, is the case of fasting glucose levels. Here the body is at a relatively static situation and the glucose levels in the blood and surrounding tissue interstitial fluid are at equilibrium. The glucose levels in the capillary blood and the interstitial fluid would be the same.

Second, there is the case of rapidly rising glucose levels such as during an oral glucose tolerance test in which a 75 g dose of glucose is rapidly ingested, or following a high caloric meal. The blood glucose level will rise rapidly as the body tries to absorb the glucose from the digestive tract. The nutrient exchange between the capillary blood and the immediately surrounding interstitial fluid is relatively fast while more distal interstitial fluid exchanges take much longer by diffusion. The best evidence is that a 4 to 8 minute time lag is typical for the glucose in the interstitial fluid in the epidermal layer to reach the same level as the capillary glucose level. This lag continues until the blood level glucose peaks and begins a relatively slow decline. The body stores the excess glucose temporarily in the interstitial fluid until the liver can process the glucose into other metabolites or eventually fat.

In the situation where the blood glucose levels are falling rapidly due to a high insulin dose, the reverse situation occurs. The blood glucose is depleted by passing through the liver and peripheral tissues while the reservoir of glucose in the skin epidermis is now back diffusing into the blood as the capillary blood glucose level falls. Again, the time difference is on the order of 4 to 8 minutes with the interstitial fluid level falling faster than the capillary blood level. Since the epidermal layer glucose is not being replenished, it falls below the capillary level and this depletion precedes the fall in capillary glucose by about 4 to 8 minutes. In the case of a diabetic patient beginning to go to hypoglycemia, measuring the glucose level in the epidermal interstitial fluid would be an advantage. In both the rapidly rising and falling blood level glucose situations, the brain is adjusting hormonal levels to try to regain the normal glucose range as rapidly as possible. In the case of severely depleted blood glucose levels, the brain adjusts glucose usage in the body to preserve the blood supply level of glucose as long as possible.

There is one clinical condition in which knowing the interstitial glucose levels can provide some critical information not presently available to physicians. In the case of severe hypoglycemia, both the tissue interstitial fluid and the blood glucose levels are very low. Conventional treatment in this emergency is to infuse glucose such that the level in the blood remains in the normal range (90 to 120 mg/dL) until the patient recovers. This can take hours. The infusion rate is not higher to prevent overshooting the target level and causing hyperglycemia with shock. If the infusion is interrupted before the interstitial fluid levels have also returned to the normal range, the blood glucose level will rapidly fall since there is no readily available source of glucose from the interstitial fluid to replenish the blood supply.

If the interstitial fluid glucose levels were monitored, the healthcare providers would know when it would be safe to stop the infusion saving patient time and money. When the interstitial fluid glucose returns to the level of the blood glucose during an infusion, the patient is well on the way to recovery. In the future, continuous monitoring of interstitial fluid glucose levels would also permit a higher level of glucose infusion without the danger of overshooting the target glucose level, thereby shortening the patient's time in the dangerous clinical condition. The very long lag time for the interstitial fluid glucose level to recover to the normal blood glucose level after a severe hypoglycemic episode is called hypoglycemic hysteresis.

From the discussion above, there are two types of glucose monitoring needs. One is for measuring glucose under the fasting condition, such as in health fairs or walk-up clinics for which a quick test is used to screen people for further testing for the diabetic condition. Since nearly 50% of Type 2 (non-insulin dependent diabetics) have not been diagnosed, a quick and painless test would be useful to identify people for further tests to catch the disease in the early stages.

The second is monitoring needs of the diagnosed diabetic, plus testing at the physician office, in hospital wards, and in the emergency room/critical care situation. Here the need is to accurately track the rapid changes in glucose level to prevent hyper and hypo glycemic episodes with their attendant complications.

The Photonic Molecular Probe which analyzes the bulk fluid properties of the sample in the light path, is useful for the fasting glucose tests. The system looks through the bulk tissue of the finger or other appropriately thin tissue and report the glucose level averaged over the bulk. Contributions from cartilage, bone, fat, ligaments, etc. would be negligible, as would the intracellular glucose levels since that level is essentially zero.

For the second situation to monitoring rapid changes of glucose level in the blood, these changes are only seen in the capillary blood and the surrounding interstitial fluid. Using the light that probes the bulk tissue of a finger would average the rapid changes of glucose in the epidermis layer over the relatively unchanged levels of glucose through the rest of the finger. A large change in epidermal glucose, reflecting the capillary blood glucose level, would not be measured if the entire finger tissue were used.

These levels are monitored using the epidermal glucose levels in the rapidly perfused tissues of the body. Since the skin layer extends at most 1.5 mm deep (including the epidermis, dermis, and underlying support tissue), the Photonic Molecular Probe need only sample at most a few hundred microns into the tissue to see the epidermal and uppermost dermis layer. The capillary bed is at the junction of the epidermis and dermis. At near infrared wavelengths, the tissue is sufficiently transparent. The measurement can be made by reflection optics, or other well-known methods to send and retrieve light a short distance into a tissue medium.

The use reflected light was to reflect the signal through a window (which complicates things a bit due to the refraction) against which the finger is pressed to give a more or less flat surface is another embodiment of the Photonic Molecular Probe. It is further possible to take advantage of what is known as frustrated interference at a near Bragg angle reflection at a refractive interface. This is a “fuzzy” waveguide effect wherein the imperfectly reflected signal picks up some information in the translucent surface of the finger.

In view of the foregoing description, numerous modifications and alternative embodiments of the invention will be apparent to those skilled in the art. Accordingly, this description is to be construed as illustrative only and is for the purpose of teaching those skilled in the art the best mode of carrying out the invention. Details of the structure may be varied substantially without departing from the spirit of the invention, and the exclusive use of all modifications which come within the scope of the appended claim is reserved.

We claim:

1. An opto-electronic device utilizing a band of partially polarized polychromatic light for quantitative analysis of a specimen containing a target molecule and a reference molecule. the device comprising:
a polarizer for producing a segmented band of partially polarized polychromatic light from the band of partially polarized polychromatic light;
a specimen cell adapted for receiving the specimen and for transporting the segmented band of partially polarized polychromatic light to the specimen;
a polarizing analyzer optically coupled to the segmented band of partially polarized polychromatic light exiting the specimen; and
comparison means for comparing the segmented band of partially polarized polychromatic light before entering the specimen with the segmented band of partially polarized polychromatic light after exiting the specimen,
wherein the target molecule and the reference molecule change ellipticity of the segmented band of partially polarized polychromatic light.

2. The device as recited in claim 1 wherein the quantitative value the target molecule in the specimen is a function of the reference molecule.

3. The device as recited in claim 2 wherein a selected optical path is used to measure the reference molecule having a predefined, known or determinable value, and a second measurement is made through the selected optical path, so that the optical distance of the selected path is corrected for by use of the first reference molecule measurement.

4. The device as recited in claim 1 wherein the polarizing analyzer is a movable polarizing analyzer.

5. The device as recited in claim 4 wherein the polarizer is a movable polarizer.

6. The device as recited in claim 5 wherein the movable polarizer is synchronized with the movable polarizing analyzer.

7. The device as recited in claim 1 further comprising a frequency filter optically coupled to the polarizer, wherein the frequency filter produces a beam of narrow frequency band of ellipitically polarized light from the band of partially polarized polychromatic light.

8. The device as recited in claim 1 further comprising a frequency filter for producing a single frequency elliptically polarized light beam from the band of partially polarized polychromatic light.

9. The device as recited in claim 1 wherein the specimen cell is implanted in a living subject.

10. The device as recited in claim 9 further comprising a medication delivery system.

11. The device as recited in claim 10 wherein the medical delivery system uses skin absorption, injection, air injection, oral application, nasal application or rectal application.

12. The device as recited in claim 1 wherein the quantitative value the target molecule is independent of deposits and contamination in the optical pathway.
